(19) United States
(12) Patent Application Publication
(10) Pub. No.: US 2004/0146426 A1
(43) Pub. Date: Jul. 29, 2004

(54) Sterilization of surfaces

(76) Inventors: Holger Biering, Grevenbroich (DE); Rudolf Glasmacher, Monheim (DE); Friedrich Von Rheinbaben, Monheim (DE)

Correspondence Address: Andrew D Sorensen, Ecolab Inc, Research and Development Center, 840 Sibley Memorial Highway, Mendota Heights, MN, 55118, US

(21) Appl. No.: 10/478,099

(22) PCT Filed: May 11, 2002
(86) PCT No.: PCT/EP02/05199

(30) Foreign Application Priority Data
May 21, 2001 (DE) 101 24 817.2

(51) Int. Cl. A61L 2/18

(52) U.S. Cl. 422/28; 422/37

(57) ABSTRACT

An agent for the treatment of medical instruments is provided which is obtainable by the artificial UV irradiation of an aqueous solution containing a chemical disinfectant, optionally together with corrosion inhibitors, buffer components, complexing agents, wetting agents and/or other auxiliary substances or active substances.

The present invention relates to an agent for the treatment of medical instruments which is obtainable by the artificial UV irradiation of a disinfectant solution, and to a method for the sterilizing treatment of medical instruments with and without sterile water rinsing. The present invention further relates to a system for the sterilizing treatment of medical instruments which consists of a UV source and a disinfectant.

U.S. Pat. No. 4,731,222 and U.S. Pat. No. 4,992,706 deal with the sterilization of medical instruments using liquid disinfectants. In this process the medical instruments are rinsed with sterile water, produced by the filtration of e.g. tap water through a sterile filter, until they are free of disinfectant residues.

U.S. Pat. No. 6,103,189 discloses a method which dispenses with sterile water as rinsing water by rinsing with a sterilizing solution which is free of non-decomposing components. According to the Example in column 5, lines 16 to 18, the conditions are to be chosen so as to achieve sterilization of the tap water used.

The state of the art methods for the sterilizing treatment of medical instruments deal substantially with the use of chemical disinfectants to destroy the relevant microorganisms. The disadvantage of the known technologies is the sometimes high expenditure associated with the time required and with the amounts of disinfectant required for a satisfactory efficacy. These large amounts of disinfectant used in the sterilizing treatment of medical instruments or for conditioning the rinsing liquid create serious problems. The greater the amount of disinfectant remaining on the surface of the medical instruments, the greater is the amount of disinfectant brought into contact with the patient on whom the medical instruments are subsequently used. On the other hand, the amount to be used cannot be arbitrarily reduced because, as also shown in the Examples of the present invention, gaps can occur in the action against important bacterial spectra, such gaps being unacceptable in practice, especially in the medical sector.

Accordingly the object of the present invention was to provide agents, methods and systems for reducing the amount of chemical disinfectant required for the sterilizing treatment and especially the amount of chemical disinfectant remaining on the medical instruments after completion of the treatment, while still achieving a sufficient efficacy against the relevant bacteria.

The present invention consequently provides agents for treating medical instruments which are obtainable by the artificial UV irradiation of an aqueous solution containing a chemical disinfectant, optionally together with corrosion inhibitors, buffer components, complexing agents, wetting agents and/or other auxiliary substances or active substances, the chemical disinfectant preferably being selected from ozone, chlorine dioxide, sodium hypochlorite, hypochlorous acid, hydrogen peroxide, organic or inorganic per acids or mixtures thereof, optionally together with other auxiliary substances or active substances and/or water.

Particularly preferred agents according to the, invention are those in which said organic per acids are selected from

a) per acids or salts of per acids of general formula I:

R²—O₂C—(CH₂)ₓ—CO₃H (I)

in which R² is hydrogen or an alkyl group having 1 to 4 carbon atoms and x is a number from 1 to 4, and/or

b) phthalimidopercarboxylic acids II, in which the percarboxylic acid moiety contains 1 to 18 carbon atoms, and/or

c) compounds of formula III:

R₁—CO₃H (III)

in which R¹ is an alkyl or alkenyl group having 1 to 18 carbon atoms.

Very particularly preferred agents according to the invention are those in which

a) the per acids of general formula I present are per acids in which R² is hydrogen or a methyl group, and/or

b) the per acids present are phthalimidopercarboxylic acids in which the percarboxylic acid moiety contains 1 to 8 carbon atoms, and/or

c) the per acids of general formula III present are per acids with an alkyl or alkenyl group having 1 to 12 carbon atoms.

In one very particularly preferred embodiment of the present invention, the per acid present in the agent according to the invention is one or more compounds selected from peracetic acid, perpropionic acid, peroctanoic acid, phthalimidoperhexanoic acid, phthalimidoperoctanoic acid, persuccinic acid, monomethyl persuccinate, perglutaric acid, monomethyl perglutarate, peradipic acid, monomethyl peradipate, persuccinic acid and monomethyl persuccinate, peracetic acid being preferred among this selection of components. In cases where the agent according to the invention is not intended for use in the final rinsing step, it preferably also contains fatty acids having 8 to 12 carbon atoms and/or anionic surfactants selected from C₈-C₁₈-alkylsulfates, C₈-C₁₈-alkyl ether sulfates, C₈-C₁₈-alkanesulfonates, C₈-C₁₈-α-olefinsulfonates, sulfonated C₈-C₁₈ fatty acids, C₈-C₁₈-alkylbenzenesulfonates, mono- and di-C₁-C₁₂-alkylsulfo-succinates, C₈-C₁₈-alkyl polyglycol ether carboxylates, C₈-C₁₈—N-acyltaurides, C₈-C₁₈—N-sarcosinates, C₈-C₁₈-alkylisethionates and mixtures of the above.

It is also preferable to obtain the agent according to the invention by the UV irradiation of a solution which, apart from water and the decomposition products of said disinfectant contained in the solution, is free of non-decomposing components. In terms of the invention, components of the agent are to be understood as meaning those which are added intentionally and remain stable under the described conditions. Accordingly, components added unintentionally, for example salts which enter the agent when using tap water, are not to be taken into account in this context. Non-decomposing components are still present in the original concentration even after a substantial period of time, for example one day or one week. In contrast thereto, according to the present invention, there are substances which gradually decompose, for example due to disproportionation or their oxidative character, and whose scission products are either less antimicrobial or less toxic than the original substance or are at least sufficiently unstable that they quickly react with other components to give decomposition products which are less antimicrobial or less toxic than the original substance. When agents or solutions free of non-decomposing ingredients are mentioned within the framework of the present invention, these basically exclude water and the decomposition products of the disinfectants contained in said agents or solutions.

In one preferred embodiment of the agent according to the invention, the solution to be treated by UV irradiation contains 0.0001 to 0.2 wt. % of disinfectant, based on the whole solution.

When using the special embodiment of an agent according to the invention which is free of non-decomposing ingredients, the solution to be treated by UV irradiation contains preferably 0.0002 to 0.01 wt. %, particularly preferably 0.001 to 0.005 and very particularly preferably not more than 0.03 wt. % of disinfectant, based on the whole solution.

The present invention also provides a method for the sterilizing treatment of microbiologically contaminated surfaces of medical instruments, wherein the surfaces of the medical instruments are brought into contact with an agent according to the invention, optionally after a cleaning step, and are then rinsed with sterile water.

The present invention also provides a method for the sterilizing treatment of microbiologically contaminated surfaces of medical instruments without rinsing with sterile water, wherein

a) an aqueous solution of a chemical disinfectant, optionally together with corrosion inhibitors, buffer components, complexing agents, wetting agents and/or other auxiliary substances or active substances, is provided,

b) the surfaces of the contaminated medical instrument are decontaminated with the solution according to a), and then

c) the surfaces of the contaminated medical instrument are treated with another aqueous solution of a liquid chemical disinfectant which is free of non-decomposing components, characterized in that the other aqueous solution used is the embodiment of the agent according to the invention, preferably with the proviso that the proportion of disinfectant present in the solution to be treated for preparation of the agent used is 0.002 to 0.01 wt. %, based on the whole solution.

The present invention also provides a method for the sterilizing treatment of microbiologically contaminated surfaces of medical instruments without rinsing with pure sterile water, wherein

a) the surfaces of the contaminated medical instrument are decontaminated with a first agent according to the invention, and then

b) the surfaces of the contaminated medical instrument are treated with another agent according to the invention, the proportion of disinfectant in the other agent according to the invention being lower than the proportion of disinfectant in the first agent according to the invention.

Particularly preferably, the proportion of disinfectant in the other agent is chosen so that, without the additional use of UV light, said proportion would be insufficient, under the chosen process conditions such as time and temperature, to destroy certain groups of pathogenic bacteria, for example mycobacteria or spores.

The present invention also provides a system for the sterilizing treatment of medical instruments, optionally without pure sterile water, which comprises

a) a UV source,

b) a chemical disinfectant selected from ozone, chlorine dioxide, sodium hypochlorite, hypochlorous acid, hydrogen peroxide, organic or inorganic per acids or mixtures thereof, optionally together with other auxiliary substances or active substances and/or water, and optionally

c) corrosion inhibitors and/or

d) buffer components and/or

e) complexing agents and/or

f) wetting agents and/or

g) other auxiliary substances or active substances.

### EXAMPLES

Procedure:

The microbiological tests were carried out within the framework of the present invention using a disinfectant solution DES1, i.e. a mixture of the following formulation: 61.1 wt. % of deionized water, 28.7 wt. % of hydrogen peroxide and 10.2 wt. % of acetic acid. The resulting proportion of peracetic acid in DES1 is 4.5 wt. %, based on the whole DES1.

The test bacteria used for the experiments were Pseudomonas aeruginosa (DSM 939) and Bacillus subtilis (ATCC 6633), spore suspension.

The water to be used was contaminated with the test bacteria in the following concentrations:

| | |
|---|---|
| Pseudomonas aeruginosa | approx. 10⁴-10⁵ CFU/ml |
| Bacillus subtilis | approx. 10³-10⁴ CFU/ml |

If the water was contaminated with only one of said bacterial species, this is stated specifically below.

With the UV apparatus switched on and heated up (lead time: 30 min), the experiment was performed first in a run with contaminated water not containing DES1 and then with contaminated water containing DES1 in an amount such that the solution in one experiment contained about 30 ppm and in another experiment about 10 ppm of peracetic acid. The apparatus was rinsed with water and disinfected after every run to prevent any residual test bacteria from falsifying the result of the next run.

The UV apparatus used was a UV apparatus suitable for the disinfection of liquid meches by the flow-through method, artificial UV-C radiation being produced by means of low-pressure mercury vapor lamps. Approx. 80% of the entire radiation from a UV lamp is at a wavelength of 254 nm (the absorption curve of DNA exhibits a maximum at 260 nm). The throughput of said UV apparatus is preferably between 640 and 800 l/h.

The actual peracetic acid concentration was determined by regular sampling using current methods. This actual peracetic acid concentration is shown in brackets in Tables 1 and 2.

The following experiments were also performed:

An experimental run with contaminated water (contaminated only with Pseudomonas aeruginosa) with the UV apparatus switched off and without adding DES1.

An experimental run with the UV apparatus switched off and with DES1 in amounts corresponding in one experiment to approx. 30 ppm and in another experiment to a concentration of approx. 10 ppm of peracetic acid in the contaminated water (Pseudomonas aeruginosa only). In these experiments the apparatus was also disinfected after each addition of DES1.

An experimental run with contaminated water (contaminated only with Pseudomonas aeruginosa) with the UV apparatus switched on but without adding DES1.

Sampling:

Sampling involved taking a sufficient amount of water and neutralizing the active substance with an inactivating solution, this being followed by membrane filtration and by the preparation of a dilution series and determination of the bacterial count.

Results:

The test bacterium Pseudomonas aeruginosa, which is also one of the most important water-borne bacteria, is markedly reduced in the apparatus in the presence of the product DES1. The concentration of DES1 required for bacterial reduction can be substantially reduced by switching on the UV apparatus. The results are shown in the Tables. By contrast, UV treatment alone effects only a slight reduction of this test bacterium.

The count of the test bacterium Bacillus subtilis is already markedly reduced by the UV treatment. DES1 alone has scarcely any effect in this case, but the bacterial reduction in the experiments with UV in the presence of DES1 is substantially greater than in the experiments with UV treatment and without DES1.

Taking the two test bacteria as a whole, the use of DES1 in combination with UV light affords a good efficacy. Such advantageous results cannot be achieved by using either DES1 or UV irradiation alone. The combination of UV light with DES1 could not be expected to produce this surprising increase in effect. It should be pointed out in particular that, by means of additional treatment with UV irradiation, it is possible substantially to reduce the amounts of DES1 required to completely destroy both test bacteria. This fact is particularly important because it enables the concentration of peracetic acid to be kept below the 50 ppm limit; experience shows that, above this level, operators are disturbed by the odor of acetic acid/peracetic acid. Apart from this, the possibility of reducing the concentration of antimicrobial substances is also welcome from the toxicological point of view. Thus the use of agents, methods and systems which adhere to the principle according to the invention ensures that the residual amount of substances which may be toxicologically critical is very low. The practical risk is consequently reduced as well.

TABLE 1
Testing bacterial reduction under the influence of
peracetic acid solution with and without UV irradiation

| Experiment set-up | Pseudomonas | Bacillus subtilis |
|---|---|---|
| Bacterial content | aeruginosa DSM 939 | (ATCC 6633), spore |
| after treatment | (K 1111) | suspension |
| Bacterial count in | 5.6 × 10⁴/ml | 6.4 × 10⁴/ml |
| the apparatus | | |
| before treatment | | |
| UV irradiation | 4.86 × 10³/ml | 9.7 × 10¹/ml |
| only | | |
| UV plus DES1 | 12 CFU/100 ml | 56 CFU/100 ml* |
| | (28 ppm of PAA) | (28 ppm of PAA) |
| DES1 without UV | 2.5 × 10⁰/ml | 7.5 × 10⁴/ml |
| | (31 ppm of PAA) | (31 ppm of PAA) |
| UV plus DES1 | 1 CFU/100 ml* | 37 CFU/100 ml* |
| | (12 ppm of PAA) | (12 ppm of PAA) |
| DES1 without UV | 5.70 × 10²/ml | 7.3 × 10⁴/ml |
| | (11.5 ppm of PAA) | (14 ppm of PAA) |

*The data “per 100 ml” relate to experimental results where only the indicated number of bacteria were detected even after processing 100 ml.

1. Use of an agent which is obtainable by the artificial UV irradiation of an aqueous solution containing a chemical disinfectant, optionally together with corrosion inhibitors, buffer components, complexing agents, wetting agents and/or other auxiliary substances or active substances for the treatment of microbiologically contaminated surfaces in particular medical instruments, without rinsing with sterile water wherein
a) said surfaces are decontaminated with the first agent defined above
b) said surfaces are treated with another agent which is free of non-decomposing components and wherein
c) the proportion of disinfectant in the other agent being lower than the proportion of disinfectant in the first agent.

2. Use according to claim 1, characterized in that the chemical disinfectant is selected from ozone, chlorine dioxide, sodium hypochlorite, hypochlorous acid, hydrogen peroxide, organic or inorganic peracids or mixtures thereof, optionally together with other auxiliary substances or active substances and/or water.

3. Use according to claim 2, characterized in that said organic peracids are selected from
a) peracids of salts of peracids of general formula
R²—O₂C—(CH₂)x—CO₃H (I)
in which R² is hydrogen or an alkyl group having 1 to 4 carbon atoms and x is a number from 1 to 4, and/or
b) phthalimidopercarboxylic acids (II), in which the percarboxylic acid moiety contains 1 to 18 carbon atoms, and/or
c) compounds of formula III:
R¹—CO₃H (III)
in which R1 is an alkyl or alkenyl group having 1 to 18 carbon atoms.

4. Use according to claim 3, characterized in that
a) the peracids of general formula I present are peracids in which R² is hydrogen or a methyl group, and/or
b) the peracids present are phthalimidopercarboxylic acids in which the percarboxylic acid moiety contains 1 to 8 carbon atoms, and/or
c) the peracids of general formula III are peracids with an alkyl or alkenyl group having 1 to 12 carbon atoms.

5. Use according to claim 4, characterized in that the peracids present are one or more compounds selected from peracetic acid, perpropionic acid, peroctanoic acid, phthalimidoperhexanoic acid, phthalimido-peroctanoic acid, persuccinic acid, monomethyl persuccinate, perglutaric acid, monomethyl perglutarate, peradipic acid, monomethyl peradipate, persuccinic acid and monomethyl persuccinate.

6. Use according to claim 5, characterized in that the peracid present is peracetic acid.

7. Use according to one or more of claims 1 to 6, characterized in that the solution to be treated by UV irradiation does not contain any non-decomposing components.

8. Use according to one or more of claims 1 to 7, characterized in that the solution to be treated by UV irradiation contains 0.0001 to 1 wt. % of disinfectant, based on the whole solution.

9. Use according to claim 8, characterized in that the solution to be treated by UV irradiation contains 0.0002 to 0.01 wt. % of disinfectant, based on the whole solution, wherein the solution is free of non-decomposing components

10. Method according to one or more of claims 1 to 9 characterized in that the proportion of disinfectant in the other agent is chosen so that, without the additional use of UV light, said proportion would be insufficient, under the chosen process conditions such as time and temperature, to destroy certain groups of pathogenic bacteria, for example mycobacteria or spores.
